(19) United States
(12) Patent Application Publication
(10) Pub. No.: US 2025/0303192 A1
(43) Pub. Date: Oct. 2, 2025

(54) LASER THERAPY DEVICE

(71) Applicant: 528 Innovations LLC, Cedar Park, TX (US)

(72) Inventors: Brandon Lee Crawford, Cedar Park, TX (US); Kevin Marshall Johnson, Cedar Park, TX (US); Philippe G. Joffrain, Cedar Park, TX (US)

(73) Assignee: 528 Innovations LLC, Cedar Park, TX (US)

(21) Appl. No.: 19/098,846

(22) Filed: Apr. 2, 2025

Related U.S. Application Data
(60) Provisional application No. 63/573,178, filed on Apr. 2, 2024.

(51) Int. Cl. A61N 5/067; A61N 5/00; A61N 5/06

(52) U.S. Cl. CPC A61N 5/067; A61N 2005/005; A61N 2005/0626; A61N 2005/0644; A61N 2005/0651; A61N 2005/0659; A61N 2005/0663

(57) ABSTRACT

A laser therapy device includes a control module having a housing, a display, a processor, and a non-transitory computer-readable medium having executable instructions encoded thereon such that, when executed, cause the processor to operate a laser module, and one or more removable laser modules configured to couple to the control module, wherein each of the one or more laser modules comprises a laser diode, and wherein the control module is configured to interchangeably receive one of the one or more laser modules. A method for providing laser treatment with a laser therapy device includes selecting, by a user, a treatment program from the display or a companion user interface coupled to the control module; and executing, by the processor, the treatment program, where the executing includes powering the one or more laser diodes and dynamically controlling a light emitted from the one or more laser diodes.

### RELATED APPLICATION

The present application claims the benefit of U.S. Provisional Patent Application No. 63/573,178, filed on Apr. 2, 2024, the entire disclosure of which is incorporated by reference herein.

### BACKGROUND

The demand for alternative medicine has been growing rapidly in recent years. Of particular interest is laser therapy, which uses focused light of varying wavelengths to treat numerous and varied conditions, such as pain management, neurological disorders, and wound healing.

### SUMMARY OF THE DISCLOSURE

Embodiments disclosed herein relate to a system for providing laser treatment, the system including a control module having a housing, a display, a processor, and a non-transitory computer-readable medium having executable instructions encoded thereon such that, when executed, cause the processor to operate a laser module, and one or more removable laser modules configured to couple to the control module, wherein each of the one or more laser modules comprises a laser diode, and wherein the control module is configured to interchangeably receive one of the one or more laser modules.

Embodiments disclosed herein also relate to a method for providing laser treatment, the method including obtaining a laser therapy device, wherein the laser therapy device includes a control module comprising a housing, a display, a processor, and a non-transitory computer-readable medium having executable instructions encoded thereon such that, when executed, cause the processor to operate a laser module; and one or more removeable laser modules configured to couple to the control module, wherein each of the one or more laser modules comprises one or more laser diodes, and wherein the control module is configured to interchangeably receive one of the one or more laser modules; selecting, by a user, a treatment program from the display or a companion user interface coupled to the control module; and executing, by the processor, the treatment program, the executing including powering the one or more laser diodes and dynamically controlling a light emitted from the one or more laser diodes.

### BRIEF DESCRIPTION OF FIGURES

FIG. 1 is a schematic of laser therapy device in accordance with one or more embodiments of the disclosure.

FIG. 2 is a close up view of the assembled laser therapy device of FIG. 1.

FIG. 3 is a perspective view of a batter module in accordance with one or more embodiments of the disclosure.

FIG. 4 is a close up view of a lower end of the assembled laser therapy device of FIG. 2.

FIG. 5 is an exploded view of a laser therapy device in accordance with one or more embodiments of the disclosure.

FIG. 6 is an exploded view of a laser module in accordance with one or more embodiments of the disclosure.

FIG. 7 is a cross-sectional view of a diffuser casing of a laser module in accordance with one or more embodiments of the disclosure.

FIG. 8 is a perspective view of a laser therapy device showing the laser module separated from the control module in accordance with one or more embodiments of the disclosure.

FIGS. 9 and 10 show removable radionics coil attachments in accordance with one or more embodiments of the disclosure.

FIG. 11 shows an end perspective view of a laser module in accordance with one or more embodiments of the disclosure.

FIG. 12 shows a companion user interface in accordance with one or more embodiments of the disclosure.

FIG. 13 shows a library screen of a companion user interface in accordance with one or more embodiments of the disclosure.

FIG. 14 shows an integrated learning screen of a companion user interface in accordance with one or more embodiments of the disclosure.

FIGS. 15-18 show examples of treatment programs in accordance with one or more embodiments of the disclosure.

FIGS. 19 and 20 show programming elements and examples thereof in accordance with one or more embodiments of the disclosure.

FIG. 21 shows a companion user interface displaying post-treatment data and journal information in accordance with one or more embodiments of the disclosure.

FIG. 22 shows a companion user interface displaying usage and health history information in accordance with one or more embodiments of the disclosure.

FIG. 23 shows a perspective view of multiple therapy devices connected together in accordance with one or more embodiments of the disclosure.

FIG. 24 shows example calculations for determining a desired distance between a diode and a diffuser in a laser module for a laser treatment device in accordance with one or more embodiments of the disclosure.

FIG. 25 shows a computing system in accordance with one or more embodiments of the disclosure.

FIG. 26 shows a computing system in accordance with one or more embodiments of the disclosure.

### DETAILED DESCRIPTION

In the following detailed description of embodiments of the disclosure, numerous specific details are set forth in order to provide a more thorough understanding of the disclosure. However, it will be apparent to one of ordinary skill in the art that the disclosure may be practiced without these specific details. In other instances, well-known features have not been described in detail to avoid unnecessarily complicating the description.

Currently, products providing laser therapy are very targeted and limited. Clinical systems are effective; however, they are too powerful and dangerous to use from home, requiring a visit to the doctor so that therapy can be administered safely by a medical professional. In addition, the clinical devices are large and expensive. Over-the-counter laser therapy products can be used from home, however, due to safety and expense, these consumer devices are too under-powered to be effective. Furthermore, the capabilities of current consumer devices are very static and generally limited in the types of treatments that can be administered.

Embodiments disclosed herein provide a system and method for safe, effective, at-home laser therapy treatment, thereby supporting both the power of a clinical device and the accessibility of an at-home experience. Through the use of interchangeable laser modules, guided treatment programs including step-by-step therapy instructions, and a companion user interface with an additional focus on education, the system and method offer targeted therapy as well as general health and wellness management. The interchangeable laser modules may provide interchangeable laser components with low and high power options and a variety of wavelengths to match treatment needs. Embodiments disclosed herein provide a laser therapy device that a dynamically changing laser light to the user during treatment following a treatment protocol. Embodiments disclosed herein may also provide access to online treatment protocols and a community of experts. Health of the user may be tracked with the disclosed system.

Embodiments described herein also provide a laser therapy device that is high power while retaining a Class 3R rating for safety across multiple wavelengths. Additionally, embodiments disclosed herein provide a laser therapy device that may include silent or low level noise cool of the laser therapy device so that the device may be handheld while also quiet.

In general, embodiments of the disclosure include a system for providing laser treatment (also referred to herein as a laser therapy device). A control module 102 serves as a base unit for the laser therapy device 100 and is configured to integrate with modular components, for example, a laser module 104 and a battery module 106. The control module 102 may include a CPU (not shown) and a display 108. One or more laser modules 104 may be interchangeably disposed onto the control module 102. Each laser module 104 includes one or more laser diodes of varying power and wavelengths that, when attached to the control module 102, are controlled by the CPU. A battery module 106 may be inserted into, or attached onto, the control module 102, thereby providing power to the laser therapy device 100. In some embodiments, a companion user interface 110 may be configured to connect to the control module 102, thereby allowing operation of the system through the user interface. As shown, the laser module 104 is coupled to a first end of the control module 102 and the battery module 106 is coupled to a second, opposite end of the control module 102. The battery module 106 may be, for example, a lithium polymer cell.

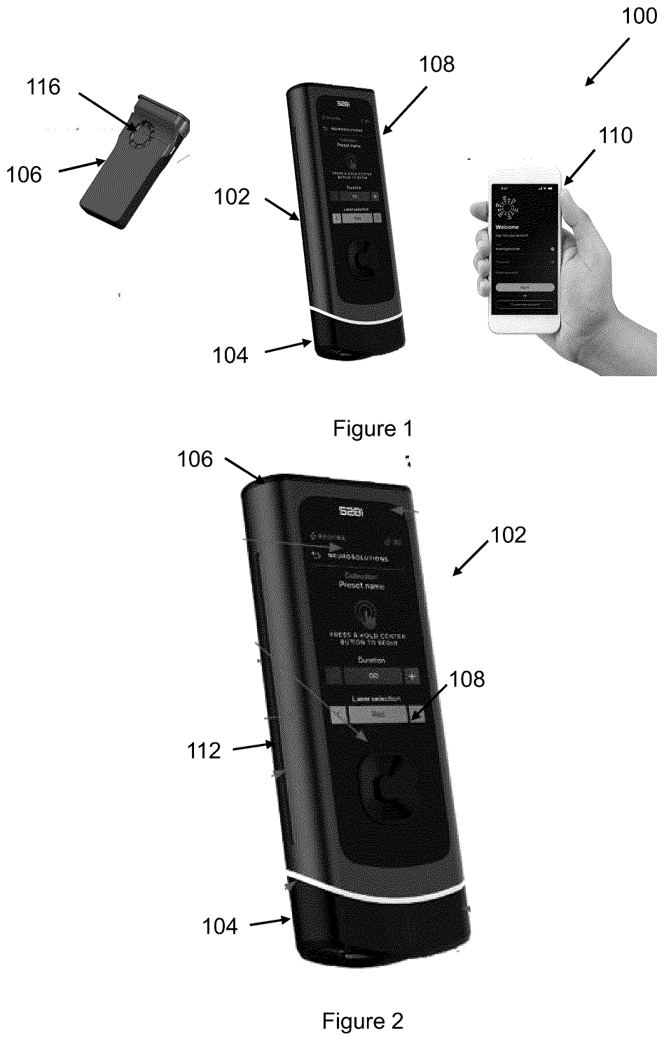

As shown in FIG. 1, a laser therapy device in accordance with embodiments of the disclosure may be described as the control module 102 combined with the battery module 106 and one of the one or more laser modules 104. The laser therapy device 100 possesses an interchangeable platform design, whereby the battery module 106 and the laser module 104 are removeable from the control module 102. Multiple laser modules 104 of different powers and wavelengths may be available and can be used interchangeably with the laser therapy device 100 by attaching the laser module 104 to the control module 102. In some embodiments, the laser module 104 may be configured to deliver electrical stimulation therapy. In one or more embodiments, the laser therapy device 100 may be handheld, or configured to fit comfortably within the hand of a user of the system. In some embodiments, the laser therapy device 100 may be wearable on the body of a user of the system, for example, as a strap, belt, or necklace. In still other embodiments, the control module 102 may be a larger fixture or free-standing machine that is configured to receive the laser module 104. Additionally, in one or more embodiments, the laser therapy device 100 may include a removable battery module 106, a battery fixed within the device, or the laser therapy device 100 may be powered directly from an electrical outlet.

The display 108 on the control module 102 allows a user of the laser therapy device 100 to navigate an interface to operate the laser therapy device 100. Examples of a display 108 may include, but are not limited to, a liquid crystal display (LCD), a plasma display, or a touchscreen. In one or more embodiments, a directional pad (114 in FIG. 4), or D-pad, or other touchpad is included on the surface of the control module 102 for a user to navigate the interface shown on the display 108. Using the control module 102 interface, the user may navigate to a treatment program based on symptoms or categories of treatment. A treatment program may be a set of step-by-step instructions which a user of the laser therapy device 100 follows to treat a specific symptom or condition with the laser therapy device 100. Based on the chosen treatment program, the laser therapy device 100 is configured to perform a proprietary set of laser beam modulations which the user can direct towards a body part as specified in the step-by-step instructions. The individual execution of a treatment program by a user of the system can be referred to as a treatment session.

The casing or housing 103 of the control module 102 may be constructed of a lightweight, durable material, such as aluminum-metal or ABS plastic. The edges of the laser therapy device 100 may be rounded to allow for a more comfortable, ergonomic experience when the device is being held or placed against the skin of a user.

In some embodiments, the control module 102 may include one or more attachment mechanisms that allow the control module 102 to be coupled to a stand or coupled to one or coupled to wearable component for a hands-free device. For example, as shown in FIG. 2, which is a close up of the assembled control module 102, laser module 104, and battery module 106, molded channels 112 may be disposed along the long edges of the control module 102 so the laser therapy device 100 can be mounted onto a stand (not shown) for the purpose of being hands-free. In other embodiments, hooks, clips, or other fasteners may be coupled to the control module 102 for coupling with, for example, a strap, a belt, or a necklace.

In one or more embodiments, the battery module 108 may be removed, or detached, from the control module 102, allowing for increased device utilization time as battery modules 106 may be swapped and recharged. For example, the battery module 106 may be inserted into a second end of the control module 102 and attached to the control module 102 by, for example, a spring-loaded latch operated by a button, one or more latches, clasps, a friction fit, or other releasable coupling mechanisms.

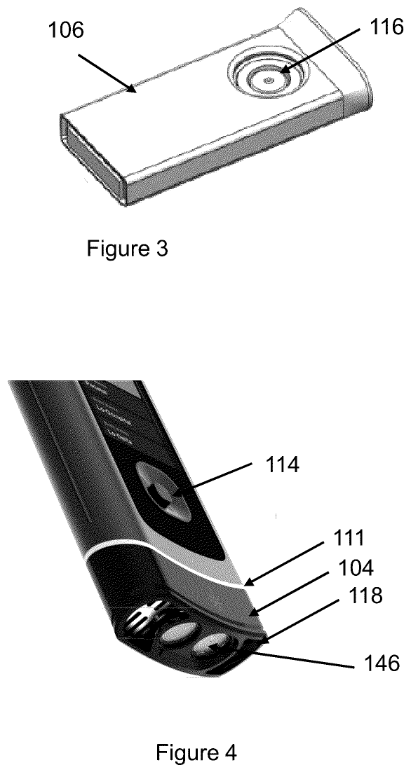

In one or more embodiments, thermal management of the laser therapy device 100 may be powered by the battery module 106 through the usage of a fan 116 integrated into the battery module 106, as shown in FIG. 3 which is an example of a battery module 106. The integrated fan 116 pulls air in through the laser module 104 and through the control module 102, thereby cooling and dissipating the heat from one or more laser diodes disposed in the laser module 104, which are described in more detail below. The air pulled through the laser module 104 by the fan 116 flows through the body of the control module 102 and exits the system through an end of the control module 102 opposite the laser module 104.

In one or more embodiments, the laser module 104 may be removed, or detached, from the control module 102, allowing a user to customize the laser therapy treatment by using laser modules 104 of different powers and wavelengths. The laser module 104 may be attached to the control module 102 by any releasable mechanism known in the art. In one embodiment, the laser module 104 may be coupled to control module 102 by a magnetic connection. In other words, a magnet fixed to the control module 102 may magnetically interact with a magnet fixed to the laser module 104, thereby removably securing the laser module 104 to the control module 102. The use of a magnetic connection may be advantageous because a magnetic connection is easy for a user to operate but strong enough to attach effectively, resulting in less wear and tear to the laser module 104 and the essential technology within it. Each type of laser module 104 may include a visual indicator to aid in visually identifying the laser module 104. For example, as shown in FIG. 4, the control module 102 may include a colored band 111 visible along the outside of the laser therapy device 100, where different colored bands 111 may indicate, for example, different laser powers, laser wavelengths, and/or uses for a particular laser module 104. In one or more embodiments, the direction of laser exposure of the laser module 104 is from a bottom of the laser therapy device 100, away from the eyes of a user, for safety reasons. Furthermore, as shown in FIG. 4, a contact surface 118 of the laser module 104 may be rounded to allow for adequate airflow when the laser therapy device 100 is pressed against the body of a user during operation of the system.

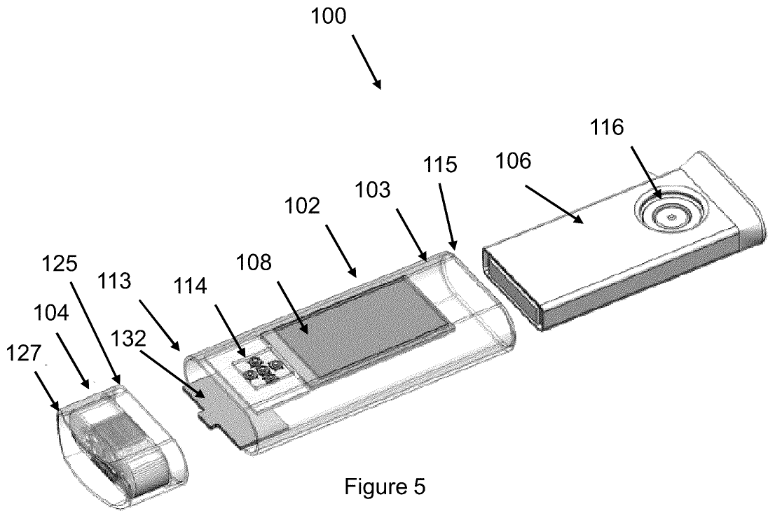

FIG. 5 shows an exploded view of laser therapy device 100. As shown, control module 102 includes a housing 103 configured to receive the laser module 104 at a first end 113 and the battery module 106 at a second end 115. As shown, the battery module 106 is configured to slide within the housing 103 of the control module 102. FIG. 5 also shows the display 108 of the control module 102 and the directional pad 114. Laser module 104 is removable and interchangeable such that different laser modules 104 having different laser specifications may be interchangeable coupled to the control module 102 depending on, for example, a specific treatment plan for a user.

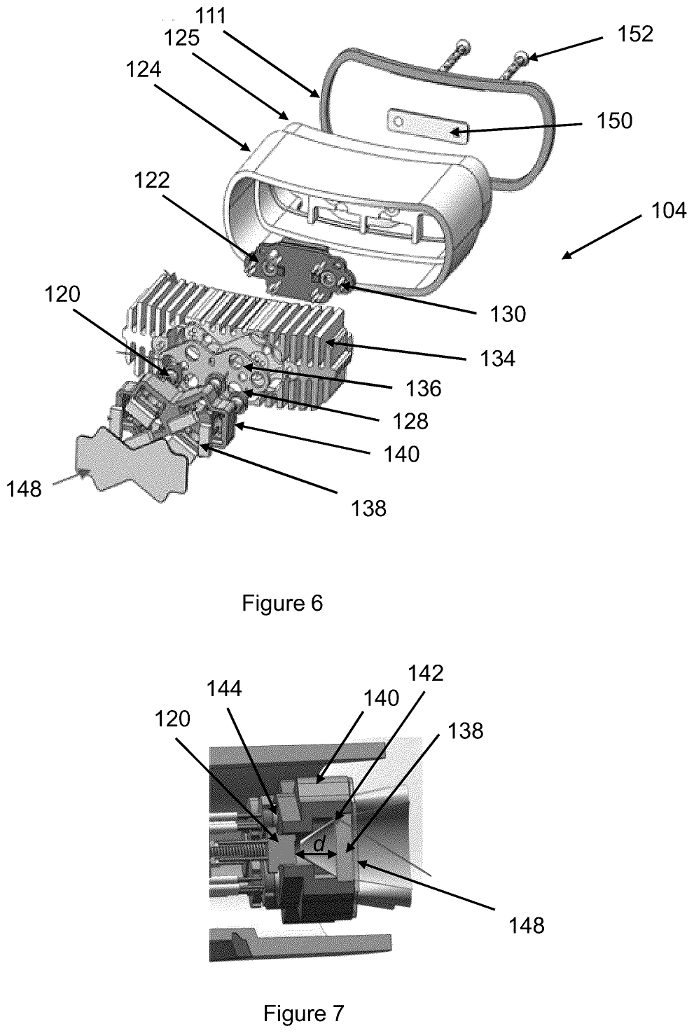

FIG. 6 shows an exploded view of an example laser module 104 in accordance with embodiments of the disclosure. As discussed, laser modules 104 may be configured to work interchangeably with the therapy laser device 100 and may be designed to produce different wavelengths and power. This breadth and flexibility allow users of the system to treat a broad spectrum of symptoms and health concerns. The laser module 104 includes a housing 124 configured to engage with a first of the control module (102, FIG. 1), to house components of the laser, and to contact a user through the contact surface (118, FIG. 4). The colored band 111 may be disposed on or around a surface of the housing 124 of the laser module 104.

In one or more embodiments, laser module 104 may include one or more laser diodes 120. In some embodiments, laser module 104 may include two laser diodes 120, four laser diodes 120, or six laser diodes 120. Each laser module 104 may produce light with different wavelengths. For example, a laser module 104 may produce light with one wavelength or light with two different wavelengths. In embodiments with multiple diodes, the diodes can be divided into different groupings of laser diodes, with each grouping working as a single set. For example, in the embodiment shown in FIG. 6, the laser module 104 may include two groupings of up to three laser diodes 120, with each grouping working together as a set. Each laser diode 120 set may be programmed to operate asynchronously from or synchronously with the other laser diode 120 set in the laser module 104. In other words, the laser therapy device 100 may be programmed such the light emitted by one set of laser diodes 120 may be started at different times or at the same time as a second set laser diodes 120. The sets of laser diodes 120 in the laser therapy device 100 may be controlled by a companion user interface (110, FIG. 1), such as device with a mobile application. The mobile application may be paired with the laser therapy device, for example by Bluetooth, to send a signal to the control module 102 to align clocks of each set of laser diodes 120 to synchronize the timing of the sets of laser diodes 120 and modulation of the laser diodes 120. Advantageously, the lasers produced by the laser module 104 in configurations disclosed herein may be rated Class 3R. Class 3R lasers are generally safe and do not require eye safety equipment such as goggles.

The following table provides example specifications for laser modules that may be used with the system:

TABLE 1
Laser Diode Specifications

| | | Diode | | |
|---|---|---|---|---|
| Wavelength | Color | Configuration | | Total Power |
| 638 nm | Red | 3 × 700 | mW | Up to 2100 mw |
| 808 nm | Infrared | 3 × 1000 | mW | Up to 3000 mw |
| 975 nm | Infrared | 3 × 500 | mW | Up to 1500 mw |
| 520 nm | Green | 2 × 300 | mW | Up to 600 mw |
| 405 nm | Violet | 2 × 350 | mW | Up to 700 mw |

As shown in FIGS. 5 and 6, a first end 125 of the housing 124 of the laser module 104 is configured to couple with the first end 113 of the control module 102. The laser module 104 also includes a printed circuit board 122. Each laser diode 120 is coupled to the printed circuit board 122 through electrical connections 126, such as pins 128 on laser diodes 120 inserted into receptacles 130 on printed circuit board 122. The printed circuit board 122 of the laser module 104 may be configured to, at least, identify the laser module 104 upon connection with the circuit board (132, FIG. 5) of the control module 102 and provide a safety mechanism to ensure a safe amount of power is being pushed from the control module 102 to the laser module 104.

Referring again to FIG. 6, the laser module 104 also includes a custom-molded heat spreader, or heat sink 134, to aid in the dissipation of heat from the laser diodes 120. The heat sink 134 may include a plate with a plurality of fins extending therefrom. In one embodiment, the heat sink 134 may include a plate with a plurality of fins extending from both sides of the plate. In some embodiments, the heat sink 134 may be formed from aluminum or copper. In some embodiments, the heat sink 134 may include a coating. The heat sink 134 may be positioned between the printed circuit board 122 of the laser module 104 and the laser diodes 120. A thermal washer (144, FIG. 7) may also be provided around the diode 120 to help distribute heat from the diode 120 to the heat sink 134. In some embodiments, a gasket 136 may be placed between the laser diodes 120 and the heat sink 134 to prevent the laser diodes 120 from shorting against the heat sink 134. The gasket 136 may be formed from any suitable material, such as foam or cork.

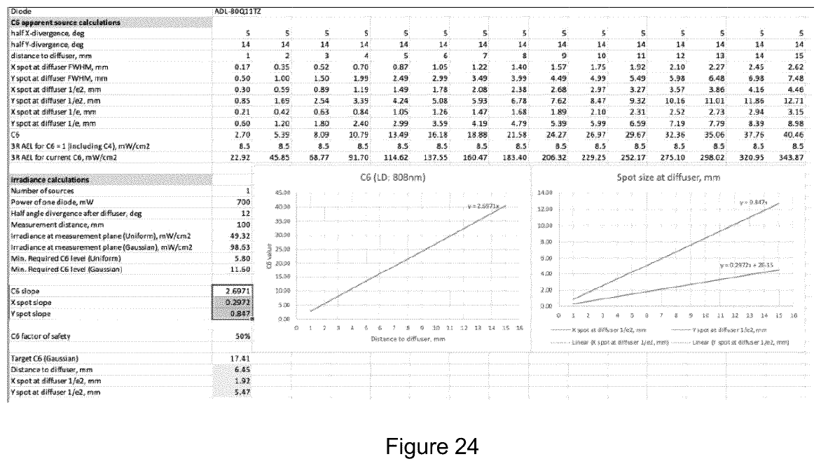

Each laser diode 120 may be paired with a laser diffuser 138. For example, in the embodiment shown in FIG. 6, there are 6 laser diodes 120 and six laser diffusers 138. A diffuser casing 140 may be used to house and position the laser diodes 120 and laser diffusers 138 in a particular configuration to ensure the correct power and safety is achieved. An example side view of a laser diode 120 and laser diffuser 138 is shown in FIG. 7 in accordance with embodiments of the disclosure. As shown in FIG. 7, the laser diffuser 138 is positioned within the casing a distance d from the laser diode 120. In some embodiments, the distance d may be between 1 mm and 30 mm. In other embodiments, the distance d may be between 1 mm and 20 mm. In other embodiments the distance d may be between 5 mm and 15 mm. FIG. 24 shows example calculations in determining a desired distance d between the laser diode 120 and the diffuser 138 to enhance safety while maintaining effectiveness of the laser light. The diffuser casing 140 may include a seat 142 or a lip or similar structure to hold the diffuser 138 in place at a predetermined distance d from the laser diode 120. A thermal washer 144 may be coupled to the diode 120 and/or the diffuser casing 140 that helps distribute heat from the diode 120 to the heat sink 134.

The laser diffuser 138 may be made of glass and positioned such that focused light from the laser diode 120 hits the laser diffuser 138, spreading the light across the glass and diffusing the light to a particular angle to produce a powerful laser that is also safe for the eye of a user. A laser therapy device 100 constructed in accordance with the present disclosure may provide a laser that has over 1000 mW of power and have a Class 3R safety rating. In some embodiments a laser therapy device 100 constructed in accordance with the present disclosure may provide a laser that has 1500 mW of power and a Class 3R safety rating.

In one embodiment, the laser diffuser 138 may be glass that may be textured or may have a film applied to at least one surface through which the light from the diode 120 passes, such that the texture or film diffuses the light uniformly. In one embodiment, the resultant light through the diffuser 138 is diffused to a uniformed angle of between 10 and 50 degrees. In other embodiments, the resultant light through the diffuser 138 is diffused to a uniformed angle of 15 degrees. In other embodiments, the resultant light through the diffuser 138 is diffused to a uniformed angle of 20 degrees. In other embodiments, the resultant light through the diffuser 138 is diffused to a uniformed angle of 25 degrees. FIG. 7 shows the light diffusing between the diode 120 and the diffuser 138 and diffusing after passing through the diffuser 138. The particular position and optical measurement (as shown, for example, in FIG. 24) of the diffuser 138 from the diode 120 allows the laser output to perform in a powerful, effective range, yet is safe enough for the eyes of a user, resulting in a Class 3R laser device.

To be characterized as a Class 3R laser device, the laser power output cannot exceed the class limit of 5 mW x C6, where C6 is the correction factor for larger source sizes, as determined through the implementation of the diffuser 138. The C6 value is calculated based on laser wavelength, divergence angle, uniformity, number of sources, placement, and/or orientation. Other considerations may be applicable for other laser classifications, without departing from the disclosure.

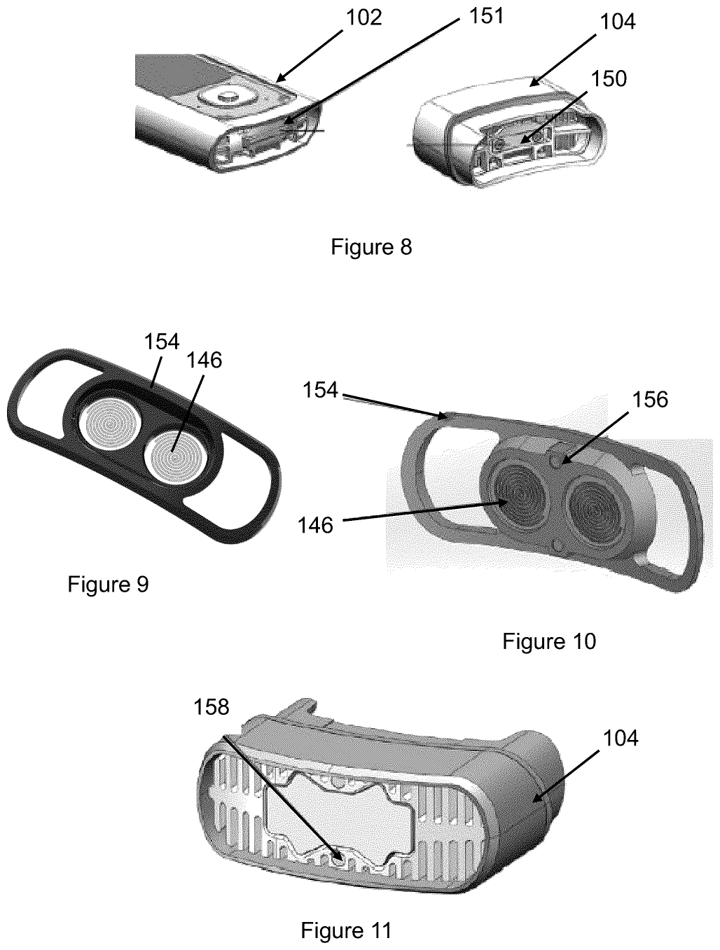

Referring to FIGS. 6 and 7, a piece of glass 148 is coupled to the diffuser casing 140 and sealed, by, for example, an adhesive, to waterproof laser therapy device 100 so that liquids do not contact the diffusers 138 or diodes 120. The laser module 104 may also include a magnet 150 to couple to a corresponding magnet 151 on the control module 102. The magnet 150 may be secured to the housing 124 of the laser module 104 by any means known in the art, such as, for example, screws 152 as shown in FIG. 6. FIG. 8 shows magnet 150 of the laser module 104 and a corresponding magnet 151 of control module 102 for providing a removable connection between the laser module 104 and the control module 102.

Referring to FIG. 4, in one or more embodiments, an integrated radionics coil 146 may optionally be coupled to the laser module 104 such that laser light from the diodes 120 hits the radionics coil 146 before contacting the user of the system. The radionics coil 146 is a passive coil (i.e., non-powered) and may be made of a conductive material such as copper, silver, or gold. The radionics coil 146 may be fixed to the laser module 104 or the radionics coil 146 may be removably coupled to the laser module 104. FIGS. 9 and 10 show examples of a removable radionics coil attachment 154 that is configured to couple to the second end 127 of the laser module 104. In one or more embodiments, the radionics coil 146 is housed in a transparent glass or plastic. The radionics coil attachment 154 may include one, two, three, or more radionics coils 146. Each radionics coil 146 may be aligned with a single diode 120 or a group of diodes 120.

As shown in FIGS. 9-11, the radionics coil attachment 154 of the radionics coil 146 may be a molded piece constructed of plastic and may include magnets 156 disposed upon the inner surface so that the radionics coil 146 may be connected to corresponding magnets 158 of the laser module 104 and may subsequently be easily removed by the user. The existence of the radionics coil 146 may serve to enhance the photon and electron delivery to the user of the system. This result is based on the photoelectric effect whereby photons interacting with a conductive metal produce more photons and electrons. The laser contacting the radionics coil 146, which may be in the form of a spiral, produces a continuous flow of electrically charged particles, amplifying the energy flowing to the user.

In one or more embodiments, laser beam modulation is controlled by the control module 102. The rate of modulation is determined by the treatment program chosen by a user of the laser therapy device 100. Each treatment program is assigned a proprietary set of laser beam modulations which the control module 102 utilizes during a treatment session. The laser beam may be dynamically modulated by modulation of the duty cycle, power level, time, pulse rate (pulse frequency) and and/or pulse width, and this modulation may continue dynamically over a period of time throughout the treatment session. In one or more embodiments, the pulse frequency of the light may be between 0.1 Hz and 100 kHz. A duty cycle of the light may be between 10% and 100%. For example, in some embodiments, the pulse frequency may be 100 kHz and a duty cycle of 50%.

Referring back to FIG. 1, in one or more embodiments, a user can operate the laser therapy device 100 through a companion user interface 110, such as a mobile application on a mobile phone or an application on a personal computer. Usage of a companion user interface 110 can provide a personalized experience for a user of the laser therapy device 100 by maintaining and analyzing user information within user profiles. The companion user interface 110 may also enhance the use of the laser therapy device 100 by providing guided treatment programs and access to educational materials. The companion user interface 110 may connect to the control unit 120 wirelessly using, for example, Bluetooth Low Energy (BLE).

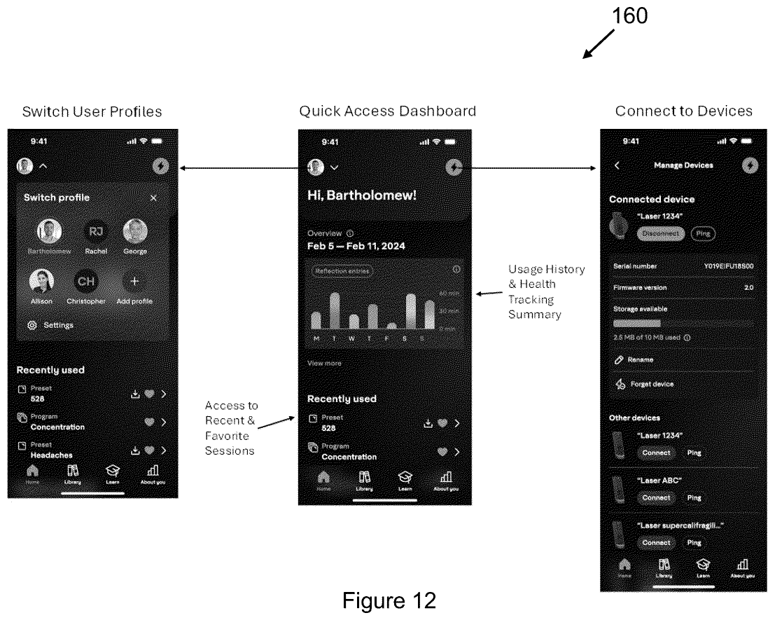

An example companion user interface 110 in the form of a mobile application 160 is shown in FIG. 12, in accordance with embodiments of the disclosure. The Home Screen, or Dashboard, can provide access to user profiles, connect to laser devices, and displays a summary overview according to a user of the system. When using the mobile application 160, multiple user profiles may be added, allowing for multiple users to operate and track usage of a single laser therapy device. The Dashboard may show a snapshot of the usage history and health tracking summary of a user, as well as provide access to recently played and favorite treatment programs.

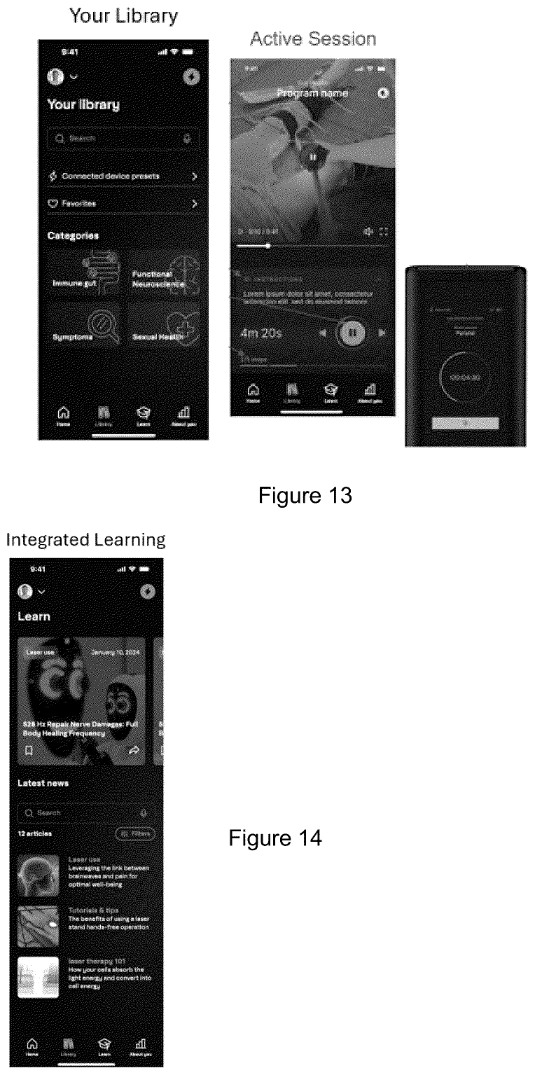

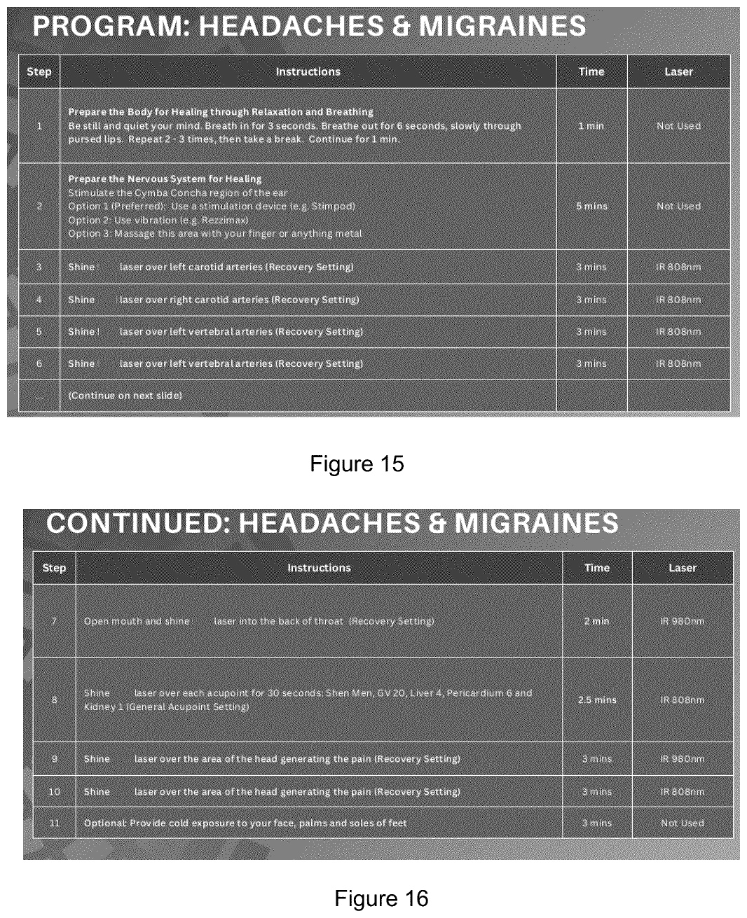

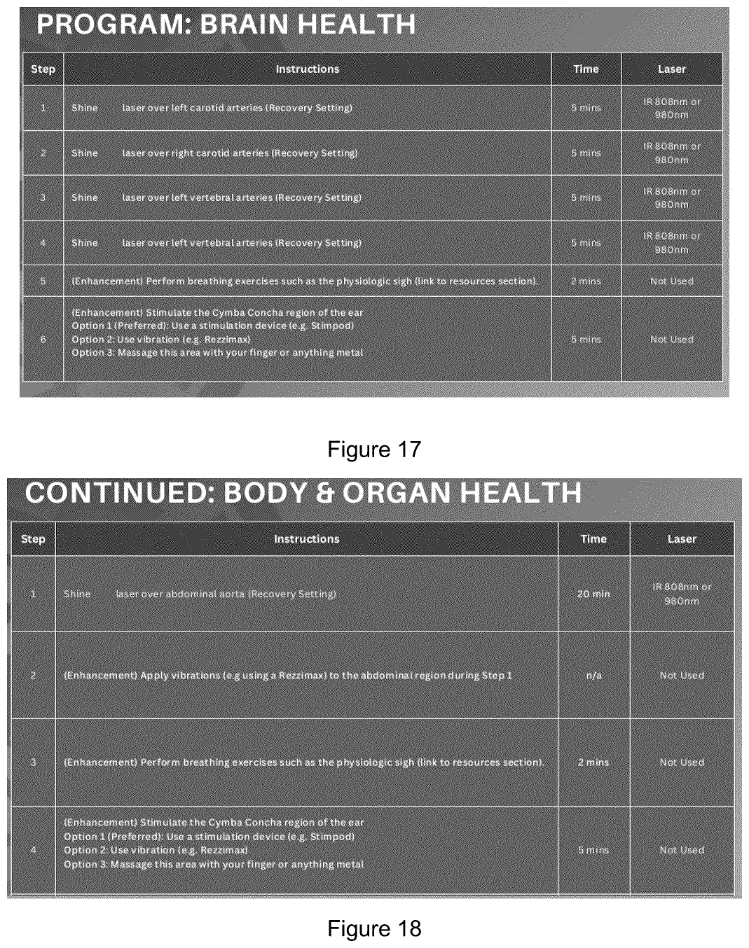

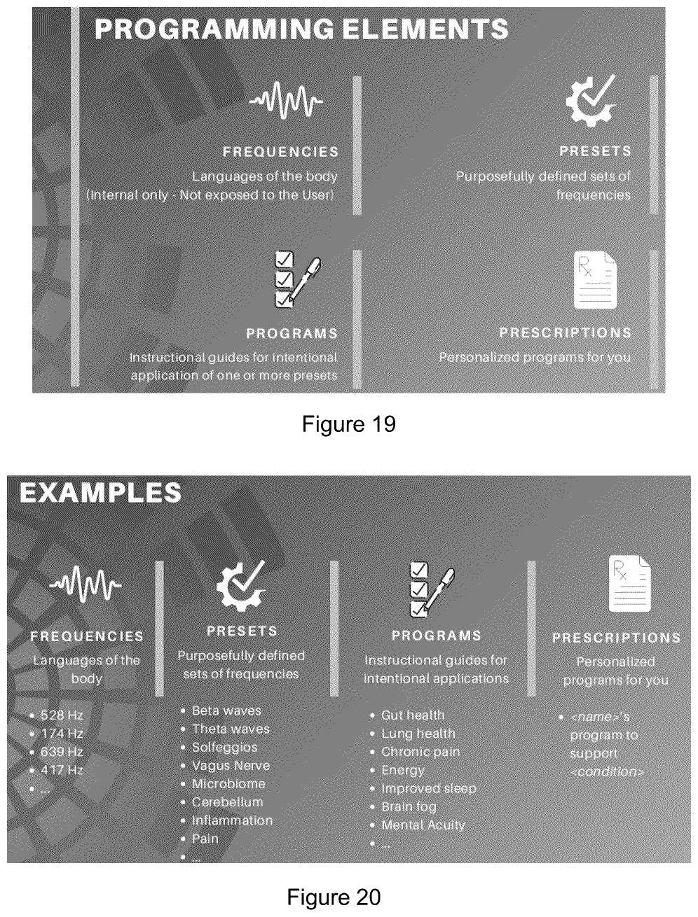

Continuing with the example mobile application 160 above, an example “Your Library” screen is shown in FIG. 13. The “Your Library” screen can give a user of the system access to treatment programs available to the system. The treatment programs may be searched for across all categories via a search bar, or alternatively a user can browse a library of treatment programs based on categories of treatment, such as “Immune Gut,” “Functional Health,” “Symptoms,” and “Sexual Health.” A treatment program can be a single-step pre-set program or a multi-step application. Once a treatment program is chosen, a treatment session may be started by the user. For each treatment program, the mobile application 160 can guide the user through the program using video instruction and/or step-by-step text instructions. Non-limiting examples of an instruction may involve a location of the body to point the laser therapy device 100, a time period which the laser therapy device 100 can be pointed to the location, or attaching a different laser module 104 to the control unit 102. Examples of a treatment program in accordance with embodiments of the disclosure are shown in FIGS. 15-18, and programming elements and examples are shown in FIGS. 19 and 20. The laser therapy device 100 can be synchronized with the treatment session, displaying information pertaining to the treatment session such as the treatment name, current step, and time remaining. Control of the treatment session (i.e. start, stop, pause, etc.) may be operated remote from the control module 102 through the mobile application 160 and/or the control module 102. This dynamic programming of the laser light allows for treatment to be tailored to the specific user's needs.

Continuing with the example mobile application 160 above, an example Integrated Learning screen is shown in FIG. 14, in one or more embodiments. The Integrated Learning screen can give a user of the system access to information such as device tutorials, educational videos, and research articles. Furthermore, in some embodiments, this information can be accessed by a user during a treatment session.

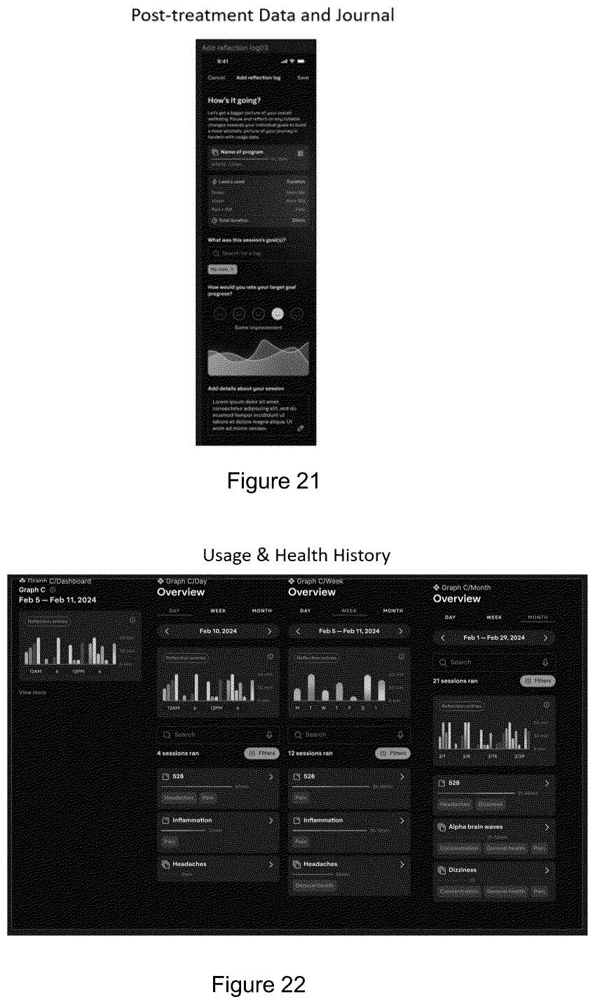

Continuing with the example mobile application 160 above, in one or more embodiments, the mobile application 160 can track and analyze the usage of a user of the system as shown in the example embodiment below. Usage history collected may be used to track the progress of a user of the system. Upon completion of a treatment session, a summary screen may be displayed showing post-treatment data such as the laser modules 104 used during the treatment session and time spent using each laser module 104. A user of the system may complete a post-treatment journal whereby the treatment session may be tagged with a descriptor, such as a symptom that was treated. Additionally, within the post-treatment journal, the progress and general wellness of a user can be rated by the user in the form of a wellness rating, also referred to as a reflection. Each wellness rating, or reflection, is correlated to a color which may subsequently be used to visually track the wellness and progress of a user of the system, as shown in FIG. 21. The information collected from a treatment session may be stored in the cloud.

Continuing with the example mobile application 160 above, in one or more embodiments, a user may view usage history as shown in the example embodiment in FIGS. 12 and 22. Usage history may be displayed over a selected period of time and filtered using, for example, the user-assigned tags or individual treatment program name. Usage history such as duration of treatment over time can be displayed in a graph summary such as a bar chart or a line graph. Results of treatment may be tracked and visualized in the graph summary by displaying the color of the wellness rating, also referred to as a reflection, in the corresponding treatment session in the graph summary. In this way, a user may visually correlate treatment progress over time using the wellness ratings, or reflections, provided by a user after each treatment session. In addition to the graph, results of treatment can be tracked and visualized by viewing the color-coded bar within each treatment program box, shown below the graph. Each treatment program box, for example “Inflammation” and “Headaches” in the figure below, lists the total time spent running an individual treatment program by a user, over one or more treatment sessions. Any descriptor tags that were added by a user in the post-treatment journal for a treatment program can be displayed in the treatment program box. The color-coded bar within each treatment program box is correlated to the wellness ratings, or reflections, that a user indicated in the post-treatment journal for a treatment program after each treatment session. As a result, the color-coded bar within each treatment program box allows a user to visualize the treatment progress and wellness over time according to an individual treatment program.

In one or more embodiments, usage and treatment results can be shared with medical professionals with the permission of the user. Additionally, with the permission of the user, usage history and treatment results can be collected and analyzed to determine health and wellness areas in which the laser therapy device 100 is producing successful results across users of the system, as well as areas that are failing to produce positive treatment results. Successful results can be reported to the U.S. Food & Drug Administration (FDA) to gain further indications for use for the benefit of marketing the system. Additionally, collected data may be used to optimize treatment programs and determine further developments for the hardware/software of the system. In one or more embodiments, collected data may be analyzed by an artificial intelligence (AI) model that can, for example, produce customized, optimized treatment programs for a user based on collected data from the treatment sessions of a user.

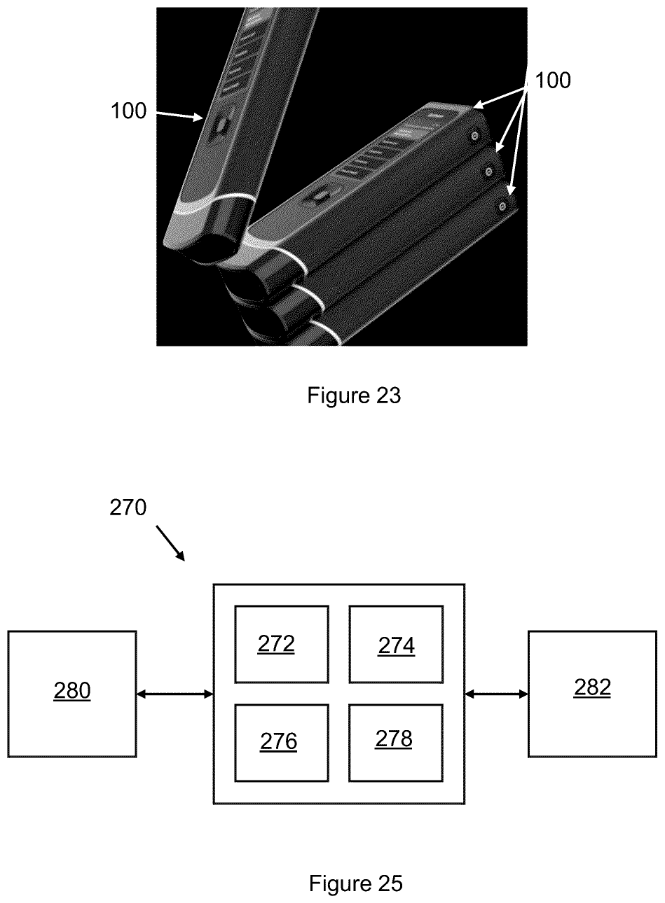

In one or more embodiments, the companion user interface 110 may connect to multiple laser therapy devices 100 at one time. When connected to multiple laser therapy devices 100, the companion user interface 110 may operate the laser therapy devices 100 synchronously as a group, or individually. When synchronized as a group, the companion user interface 110 may align the laser pulse output of each laser therapy device 100. The devices may be physically attached to each other, or stacked, as shown in the FIG. 23. The laser therapy devices 100 may be attached to each other may be, for example, by magnets, adhesive, clips, latches, locks, or other mechanical means known in the art. When stacked, the laser therapy devices 100 may be configured to leave an offset of space between each laser therapy device 100 to allow for flow of air between each of the laser therapy devices 100.

One or more embodiments of the laser therapy device 100 may be implemented on a computing system. Any combination of mobile, desktop, server, router, switch, embedded device, or other types of hardware may be used. For example, as shown in FIG. 25, the computing system 270 may include one or more computer processors 272, a non-persistent storage 274 (for example, volatile memory, such as random access memory (RAM), cache memory), persistent storage 276 (for example, a hard disk, an optical drive such as a compact disk (CD) drive or digital versatile disk (DVD) drive, a flash memory, etc.), a communication interface 278 (for example, Bluetooth interface, infrared interface, network interface, optical interface, etc.), and numerous other elements and functionalities.

The computer processor(s) 272 may be an integrated circuit for processing instructions. For example, the computer processor(s) 272 may be one or more cores or micro cores of a processor. The computing system 270 may also include one or more input devices 280, such as a touchscreen, keyboard, mouse, microphone, touchpad, electronic pen, or any other type of input device.

The communication interface 278 may include an integrated circuit for connecting the computing system 270 to a network (not shown) (for example, a local area network (LAN), a wide area network (WAN) such as the Internet, mobile network, or any other type of network) and/or to another device, such as another computing device.

Further, the computing system 270 may include one or more output devices 282, such as a screen (for example, a liquid crystal display (LCD), a plasma display, touchscreen, cathode ray tube (CRT) monitor, projector, or other display device), a printer, external storage, or any other output device. One or more of the output devices 282 may be the same or different from the input device(s) 280. The input and output device(s) may be locally or remotely connected to the computer processor(s) 272, non-persistent storage 274, and persistent storage 276. Many different types of computing systems exist, and the input and output device(s) may take other forms.

Software instructions in the form of computer readable program code to perform embodiments of the disclosure may be stored, in whole or in part, temporarily or permanently, on a non-transitory computer readable medium such as a CD, DVD, storage device, a diskette, a tape, flash memory, physical memory, or any other computer readable storage medium. Specifically, the software instructions may correspond to computer readable program code that, when executed by a processor(s), is configured to perform one or more embodiments of the disclosure.

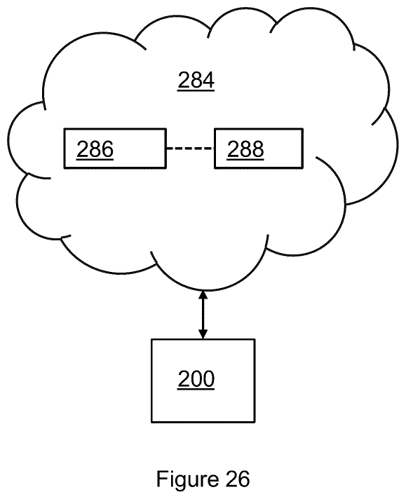

The computing system 270 in FIG. 25 may be connected to or be a part of a network. For example, as shown in FIG. 26, the network 284 may include multiple nodes (for example, a first node 286, and a second node 288). Each node may correspond to a computing system, such as the computing system 270 shown in FIG. 25, or a group of nodes combined may correspond to the computing system 270 shown in FIG. 25. By way of an example, embodiments of the disclosure may be implemented on a node of a distributed system that is connected to other nodes. By way of another example, embodiments of the disclosure may be implemented on a distributed computing system having multiple nodes, where each portion of the disclosure may be located on a different node within the distributed computing system. Further, one or more elements of the computing system 270 may be located at a remote location and connected to the other elements over a network.

The nodes (for example, nodes 286, 288) in the network 284 may be configured to provide services for a user device 200, such as laser therapy device 100. For example, the nodes may be part of a cloud computing system. The nodes may include functionality to receive requests from the laser therapy device 100 and transmit responses to the laser therapy device 100. The laser therapy device 100 may include a computing system, such as the computing system 270 shown in FIG. 25. Further, the laser therapy device 100 may include and/or perform all or a portion of one or more embodiments of the disclosure.

In one or more embodiments, a control module of a laser therapy device in accordance with embodiments disclosed herein includes a computing system as described. For example, the control module 102 may include a processor, and a non-transitory computer-readable medium having executable instructions encoded thereon such that, when executed, cause the processor to operate a laser module. The processor may be configured to control and modulate a pulse of a laser emitted from one or more laser diodes 120. For example, the processor may be configured to execute a set of instructions stored on the non-transitory computer-readable medium to modulate or change a width, a frequency, or a duty cycle of the laser light emitted from the diode based on an electrical signal send to the diode from the processor.

In one embodiment, a laser therapy device 100 may include a control module 102 comprising a housing, a display, a processor, and a non-transitory computer-readable medium having executable instructions encoded thereon such that, when executed, cause the processor to operate the laser therapy device. In this embodiment, the control module 102 may also include or house one or more laser diodes 120 such that the laser light from the laser diodes 120 is emitted directly from the control module 102. In this embodiment, the control module 102 may house or include the various laser module components described above with respect to FIGS. 6 and 7, without the need for a separate laser module 104. In other words, the housing 103 of the control module 102 may house all the components disclosed above as housed within the laser module 104 housing 124 without the need for a separate housing 124. A radionics coil 146 may be coupled directly to the control module 102 via a radionics coil attachment 154 in the same way describe with respect to attaching the radionics coil attachment 154 to the laser module 104.

In some embodiments, a laser therapy device may include a control module 102 comprising a housing, a display, a processor, and a non-transitory computer-readable medium having executable instructions encoded thereon such that, when executed, cause the processor to operate the laser therapy device 100. In this embodiment, the control module 102 may also include or house one or more laser diodes 120 such that the laser light from the laser diodes 120 is emitted directly from the control module 102. In this embodiment, the control module 102 may house or include the various laser module components described above with respect to FIGS. 6 and 7, without the need for a separate laser module 104. Thus, the control module 102 may include or house, for example, a heat sink 134, a circuit board 122 and one or more laser diodes 120 coupled to the circuit board 122, a diffuser casing 140, one or more diffusers 138 such that each diode has a diffuser 138 (or such a diffuser is placed over each diode, for example, one diffuser may be placed over two or more diodes), and a safety glass. These laser components may be located within the housing of the control module 102 and proximate the first end of the control module 102 and configured to provide a laser light through the end of the control module 102 for application to a user.

In some embodiments, a laser therapy device may include a control module 102 comprising a housing, a display, a processor, and a non-transitory computer-readable medium having executable instructions encoded thereon such that, when executed, cause the processor to operate the laser therapy device 100. The laser therapy device 100 may include laser components within the housing 103 of the control module 102 or in a removable laser module 104, as described above. The laser therapy device 100 provides dynamic control of the laser light as described above with respect to controlling, synchronously or asynchronously, the pulse of the one or more diodes 120. The laser therapy device 100 also (with or without dynamic control of the laser light) includes a radionics coil 146 coupled to the laser therapy device 100 directly to the control module 102 housing 103.

In some embodiments, a laser therapy device may include a control module comprising a housing, a display, a processor, and a non-transitory computer-readable medium having executable instructions encoded thereon such that, when executed, cause the processor to operate the laser therapy device as disclosed. The laser therapy device also includes a mobile application, as described herein, so that, for example, different treatment options may be selected from a companion user interface and so that the laser therapy device may be controlled remotely according to instructions provided by the mobile application or stored within the control module.

The various features of the laser therapy device disclosed herein, and various embodiments disclosed herein may be combined in various ways. For example, in some embodiments, a laser therapy device may include one or more of a removable laser module, a radionics coil, dynamically controlled laser light, thermal cooling of the laser, and a mobile app for controlling one or more laser therapy devices.

Although only a few example embodiments have been described in detail above, those skilled in the art will readily appreciate that many modifications are possible in the example embodiments without materially departing from this invention. Accordingly, all such modifications are intended to be included within the scope of this disclosure as defined in the following claims.

What is claimed:

1. A system for providing laser treatment, the system comprising:
a control module comprising a housing, a display, a processor, and a non-transitory computer-readable medium having executable instructions encoded thereon such that, when executed, cause the processor to operate a laser module; and
one or more removable laser modules configured to couple to the control module, wherein each of the one or more laser modules comprises a laser diode, and wherein the control module is configured to interchangeably receive one of the one or more laser modules.

2. The system of claim 1, further comprising a battery module configured to couple to the control module, thereby providing an energy source to the control module.

3. The system of claim 1, further comprising a companion user interface configured to connect to the control module.

4. The system of claim 1, wherein the system including the laser diode produces a class 3R laser.

5. The system of claim 1, wherein the processor is configured to dynamically control the laser light emitted by the laser diode.

6. The system of claim 5, wherein the processor is configured to dynamically control the laser light emitted by the laser diode by modulating a pulse of a laser emitted from the diode.

7. The system of claim 6, wherein the pulse is modulated by adjusting a width, a frequency, or a duty cycle of the laser light.

8. The system of claim 2, wherein the battery module further comprises a fan.

9. The system of claim 8, wherein the fan is configured to draw a flow of air in through the laser module, through the housing of the control module, and out through the fan.

10. The system of claim 1, wherein the system is configured to be handheld.

11. The system of claim 1, further comprising a radionics coil coupled to the laser module.

12. The system of claim 11, wherein the radionics coil is removable coupled to the laser module.

13. The system of claim 1, wherein two or more systems are physically coupled to each other.

14. The system of claim 13, further comprising a companion user interface, wherein the companion user interface is configured to synchronously and/or asynchronously control the two or more systems.

15. A method for providing laser treatment, the method comprising:
obtaining a laser therapy device, wherein the laser therapy device comprises:
a control module comprising a housing, a display, a processor, and a non-transitory computer-readable medium having executable instructions encoded thereon such that, when executed, cause the processor to operate a laser module; and
one or more removeable laser modules configured to couple to the control module, wherein each of the one or more laser modules comprises one or more laser diodes, and wherein the control module is configured to interchangeably receive one of the one or more laser modules;
selecting, by a user, a treatment program from the display or a companion user interface coupled to the control module; and
executing, by the processor, the treatment program, the executing including powering the one or more laser diodes and dynamically controlling a light emitted from the one or more laser diodes.

16. The method of claim 15, wherein executing the treatment program further comprises modulating a pulse from the one or more laser diodes of the laser module.

17. The method of claim 16, wherein modulating the pulse includes adjusting a width, frequency, and/or duty cycle of light emitted from the one or more laser diodes.

18. The method of claim 16, further comprising directing the laser therapy device to an area to be treated, such that the modulated pulse contacts the area to be treated.

19. The method of claim 15, wherein the laser therapy device further includes a battery module configured to couple to the control module, thereby providing an energy source to the control module.

20. The method of claim 15, further comprising:
connecting the laser therapy device to a second laser therapy device via a companion user interface; and
controlling each of the laser therapy device and the second laser therapy device asynchronously and/or synchronously via the companion user interface and the processor.
